(19) United States
(12) Patent Application Publication
(10) Pub. No.: US 2024/0422428 A1
(43) Pub. Date: Dec. 19, 2024

(54) MEDICAL IMAGE PROCESSING DEVICE AND MEDICAL IMAGE PROCESSING SYSTEM

(71) Applicant: Sony Olympus Medical Solutions Inc., Tokyo (JP)

(72) Inventor: Hiroshi FUKUDA, Tokyo (JP)

(73) Assignee: Sony Olympus Medical Solutions Inc., Tokyo (JP)

(21) Appl. No.: 18/669,559

(22) Filed: May 21, 2024

(30) Foreign Application Priority Data
Jun. 15, 2023 (JP) 2023-098312

(51) Int. Cl. H04N 23/667; A61B 1/00; A61B 5/00

(52) U.S. Cl. CPC H04N 23/667; A61B 1/00009; A61B 5/0071

(57) ABSTRACT

A medical image processing device includes: a plurality of storage units each configured to store a value related to an image processing parameter; and an image processing unit configured to perform image processing based on the image processing parameter determined by selecting at least one of the plurality of storage units according to a timing at which types of a plurality of types of acquired medical images are switched.

### CROSS-REFERENCE TO RELATED APPLICATION

This application claims priority from Japanese Application No. 2023-098312, filed on Jun. 15, 2023, the contents of which are incorporated by reference herein in its entirety.

### BACKGROUND

The present disclosure relates to a medical image processing device and a medical image processing system.

In the related art, an image processing device that processes a plurality of types of images having different numbers of imaging pixels or the like obtained by imaging is known (for example, see JP 2013-123172 A).

The image processing device described in JP 2013-123172 A includes a register in which an image processing parameter corresponding to a type of an image is set, and an image processing unit that performs image processing on the image using the image processing parameter set in the register. Then, the image processing device sets the image processing parameter corresponding to the type of the image in the register by software processing according to a timing at which the type of the image is switched.

### SUMMARY

In the image processing device described in JP 2013-123172 A, since the image processing parameter is set in the register by software processing, there is a problem that, for example, in a case where a switching rate of the type of the image is every frame, the load of the software increases.

Therefore, there is a demand for a technique capable of reducing the load of software when image processing is sequentially performed on a plurality of types of acquired images using image processing parameters corresponding to the types of the images for each of the images.

According to one aspect of the present disclosure, there is provided a medical image processing device including: a plurality of storage units each configured to store a value related to an image processing parameter; and an image processing unit configured to perform image processing based on the image processing parameter determined by selecting at least one of the plurality of storage units according to a timing at which types of a plurality of types of acquired medical images are switched.

According to another aspect of the present disclosure, there is provided a medical image processing system including: an imaging device configured to sequentially output a plurality of types of medical images obtained by imaging; and an image processing device configured to process the plurality of types of medical images sequentially output from the imaging device, wherein the image processing device includes a plurality of storage units each configured to store a value related to an image processing parameter, and an image processing unit configured to perform image processing based on the image processing parameter determined by selecting at least one of the plurality of storage units according to a timing at which the types of the plurality of types of acquired medical images are switched.

### BRIEF DESCRIPTION OF THE DRAWINGS

FIG. 1 is a diagram illustrating a configuration of an image processing system according to an embodiment;

FIG. 2 is a block diagram illustrating a configuration of a camera head and a control device;

FIG. 3 is a diagram for describing a function of a processing module;

FIG. 4 is a diagram for describing the function of the processing module;

FIGS. 5A to 5D are diagrams for describing a function of a selector in a first mode;

FIG. 6 is a diagram for describing a function of a selector in a second mode;

FIG. 7 is a diagram illustrating a first modification of the embodiment;

FIG. 8 is a diagram illustrating a second modification of the embodiment;

FIGS. 9A to 9D are diagrams for describing the second modification of the embodiment;

FIG. 10 is a diagram for describing a third modification of the embodiment;

FIG. 11 is a diagram for describing a fourth modification of the embodiment;

FIG. 12 is a diagram for describing a fifth modification of the embodiment;

FIG. 13 is a diagram for describing a sixth modification of the embodiment; and

FIG. 14 is a diagram for describing a seventh modification of the embodiment.

### DETAILED DESCRIPTION

Hereinafter, a mode (hereinafter, embodiment) for carrying out the present disclosure will be described with reference to the drawings. Note that the present disclosure is not limited by the embodiment described below. Furthermore, in the description of the drawings, the same portions are denoted by the same reference numerals.

### Schematic Configuration of Image Processing System

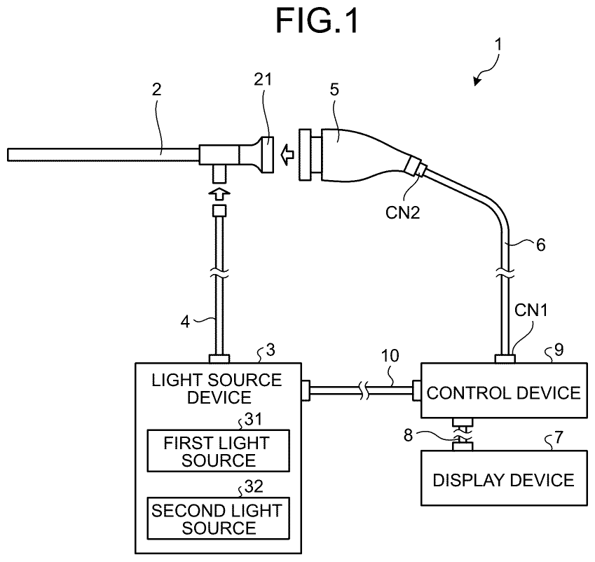

FIG. 1 is a diagram illustrating a configuration of an image processing system 1 according to an embodiment.

The image processing system 1 according to the present embodiment is a system that is used in the medical field and observes the inside of a living body. As illustrated in FIG. 1, the image processing system 1 includes an insertion unit 2, a light source device 3, a light guide 4, a camera head 5, a first transmission cable 6, a display device 7, a second transmission cable 8, a control device 9, and a third transmission cable 10.

In the present embodiment, the insertion unit 2 includes a rigid endoscope. That is, the insertion unit 2 has an elongated shape that is entirely rigid or partially soft and rigid in the remaining portion, and is inserted into the living body. An optical system (not illustrated) configured using one or a plurality of lenses and configured to collect light (subject image) from a subject is provided in the insertion unit 2.

The light source device 3 is connected to one end of the light guide 4, and supplies light to irradiate the inside of the living body to the one end of the light guide 4 under the control of the control device 9. As illustrated in FIG. 1, the light source device 3 includes a first light source 31 and a second light source 32.

The first light source 31 emits light in a first wavelength band. In the present embodiment, first light source 31 includes a light emitting diode (LED) that emits white light (light in a first wavelength band).

The second light source 32 emits light of a second wavelength band different from the first wavelength band. In the present embodiment, second light source 32 includes a semiconductor laser that emits near-infrared excitation light (light in a second wavelength band) in a near-infrared wavelength band.

The near-infrared excitation light emitted by the second light source 32 is excitation light that excites a fluorescent substance such as indocyanine green. When excited by the near-infrared excitation light, the fluorescent substance such as indocyanine green emits fluorescence having a central wavelength on a longer wavelength side than a central wavelength of a wavelength band of the near-infrared excitation light.

In the present embodiment, the light source device 3 is configured separately from the control device 9, but the present disclosure is not limited thereto, and a configuration provided in the same casing as the control device 9 may be adopted.

One end of the light guide 4 is detachably connected to the light source device 3, and the other end thereof is detachably connected to the insertion unit 2. Then, the light guide 4 transmits light (white light or near-infrared excitation light) supplied from the light source device 3 from one end to the other end, and supplies the light to the insertion unit 2. The light (white light or near-infrared excitation light) supplied to the insertion unit 2 is emitted from the distal end of the insertion unit 2 and emitted into the living body. When the white light is emitted into the living body, the white light reflected in the living body is condensed by the optical system in the insertion unit 2. Hereinafter, for convenience of description, the white light condensed by the optical system in the insertion unit 2 is referred to as a first subject image. In addition, in a case where the near-infrared excitation light is emitted into the living body, the near-infrared excitation light reflected in the living body and a fluorescent substance such as indocyanine green accumulated at a lesion in the living body are excited, and fluorescence emitted from the fluorescent substance is collected by the optical system in the insertion unit 2. In the following description, for convenience of explanation, the near-infrared excitation light and the fluorescence collected by the optical system in the insertion unit 2 will be referred to as a second subject image.

The camera head 5 corresponds to an imaging device according to the present disclosure. The camera head 5 is detachably connected to a proximal end (eyepiece unit 21 (FIG. 1)) of the insertion unit 2. Then, under the control of the control device 9, the camera head 5 captures the first subject image (white light) and the second subject image (near-infrared excitation light and fluorescence) condensed by the insertion unit 2 to generate an image signal (hereinafter, referred to as a captured image).

Note that a detailed configuration of the camera head 5 will be described in “Configuration of Camera Head” described later.

One end of the first transmission cable 6 is detachably connected to the control device 9 via a connector CN1 (FIG. 1), and the other end thereof is detachably connected to the camera head 5 via a connector CN2 (FIG. 1). Then, the first transmission cable 6 transmits a captured image and the like output from the camera head 5 to the control device 9, and transmits a control signal, a synchronization signal, a determination signal, a clock, power, and the like output from the control device 9 to the camera head 5.

Note that, in the transmission of the captured image and the like from the camera head 5 to the control device 9 via the first transmission cable 6, the captured image and the like may be transmitted as an optical signal or may be transmitted as an electric signal. The same applies to transmission of a control signal, a synchronization signal, a determination signal, and a clock from the control device 9 to the camera head 5 via the first transmission cable 6.

The display device 7 includes a display using liquid crystal, organic electro luminescence (EL), or the like, and displays an image based on a video signal from the control device 9 under the control of the control device 9.

One end of the second transmission cable 8 is detachably connected to the display device 7, and the other end thereof is detachably connected to the control device 9. Then, the second transmission cable 8 transmits a video signal processed by the control device 9 to the display device 7.

The control device 9 corresponds to an image processing device according to the present disclosure. The control device 9 includes a central processing unit (CPU), a field-programmable gate array (FPGA), and the like, and integrally controls operations of the light source device 3, the camera head 5, and the display device 7.

Note that a detailed configuration of the control device 9 will be described in “Configuration of Control Device” described later.

One end of the third transmission cable 10 is detachably connected to the light source device 3, and the other end thereof is detachably connected to the control device 9. Then, the third transmission cable 10 transmits the control signal from the control device 9 to the light source device 3.

### Configuration of Camera Head

Next, a configuration of the camera head 5 will be described.

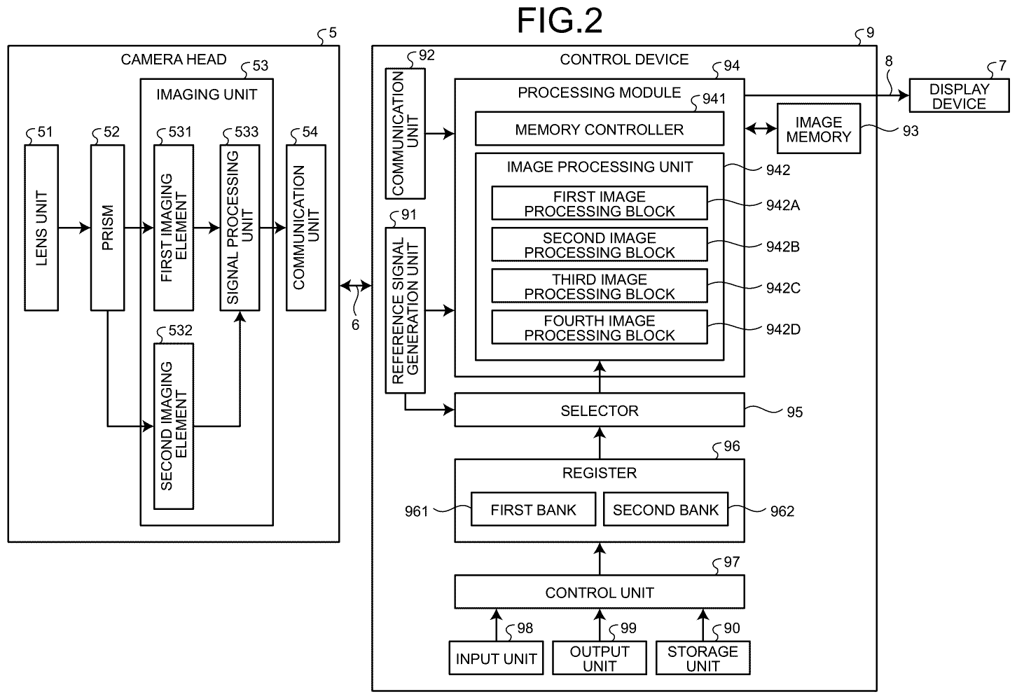

FIG. 2 is a block diagram illustrating a configuration of the camera head 5 and the control device 9.

As illustrated in FIG. 2, the camera head 5 includes a lens unit 51, a prism 52, an imaging unit 53, and a communication unit 54.

The lens unit 51 includes one or a plurality of lenses. Then, the lens unit 51 forms the first subject image (white light) condensed by the insertion unit 2 on the imaging surface of the first imaging element 531 (FIG. 2), and forms the second subject image (near-infrared excitation light and fluorescence) condensed by the insertion unit 2 on the imaging surface of the second imaging element 532 (FIG. 2).

The prism 52 separates the first subject image (white light) and the second subject image (near-infrared excitation light and fluorescence) through the lens unit 51. Then, the prism 52 advances the first subject image (white light) toward the first imaging element 531. The prism 52 also causes the second subject image (near-infrared excitation light and fluorescence) to travel toward the second imaging element 532.

Then, the imaging unit 53 captures an image inside the living body under the control of the control device 9. As illustrated in FIG. 2, the imaging unit 53 includes the first imaging element 531, the second imaging element 532, and a signal processing unit 533.

The first and second imaging elements 531 and 532 include a charge coupled device (CCD), a complementary metal oxide semiconductor (CMOS), or the like that receives incident light and converts the light into an electrical signal.

Then, the first imaging element 531 captures the first subject image (white light) via the prism 52 under the control of the control device 9. Hereinafter, for convenience of description, a captured image generated by capturing the first subject image (white light) by the first imaging element 531 will be referred to as a normal light image. In the present embodiment, first imaging element 531 has a number of pixels capable of imaging at a resolution of 4K. That is, the normal light image is a 4K image having 4K pixels.

Furthermore, the second imaging element 532 captures the second subject image (near-infrared excitation light and fluorescence) via the prism 52 under the control of the control device 9. Hereinafter, for convenience of description, a captured image generated by capturing the second subject image (near-infrared excitation light and fluorescence) by the second imaging element 532 is referred to as a fluorescence image. In the present embodiment, the second imaging element 532 has the number of pixels that can be imaged in the HD resolution. That is, the fluorescence image is an HD image in which the number of pixels is HD.

Note that an excitation light cut filter that removes only at least a part of the near-infrared excitation light traveling toward the second imaging element 532 may be disposed on the front stage side of the optical path of the second imaging element 532.

As described above, the camera head 5 includes the first and second imaging elements 531 and 532 that output different types of images (normal light image (4K image), fluorescence image (HD image)). That is, the plurality of types of images according to the present disclosure corresponds to a normal light image (4K image) and a fluorescence image (HD image) having mutually different image sizes.

Under the control of the control device 9, the signal processing unit 533 performs signal processing on the captured images (analog signals) generated by the first and second imaging elements 531 and 532 and outputs the captured images (digital signals).

For example, the signal processing unit 533 performs processing of removing reset noise, processing of multiplying an analog gain for amplifying the analog signal, and signal processing such as A/D conversion on the captured images (analog signals) generated by the first and second imaging elements 531 and 532.

The communication unit 54 functions as a transmitter that transmits captured images sequentially output from the imaging unit 53 to the control device 9 via the first transmission cable 6. The communication unit 54 includes, for example, a high-speed serial interface that communicates a captured image with the control device 9 via the first transmission cable 6 at a transmission rate of 1 Gbps or more.

### Configuration of Control Device

Next, a configuration of the control device 9 will be described with reference to FIG. 2.

As illustrated in FIG. 2, the control device 9 includes a reference signal generation unit 91, a communication unit 92, an image memory 93, a processing module 94, a selector 95, a register 96, a control unit 97, an input unit 98, an output unit 99, and a storage unit 90.

The reference signal generation unit 91 generates a signal serving as a reference necessary for the operations of the camera head 5 and the control device 9. Then, the reference signal generation unit 91 outputs the reference signal to the camera head 5, the processing module 94, the selector 95, and the like. The reference signal includes a vertical synchronization signal and a determination signal.

Note that details of the determination signal will be described in “Function of Selector” described later.

The communication unit 92 functions as a receiver that receives captured images sequentially output from the camera head 5 (communication unit 54) via the first transmission cable 6. The communication unit 92 includes, for example, a high-speed serial interface that communicates the captured images with the communication unit 54 at a transmission rate of 1 Gbps or more.

The image memory 93 includes, for example, a dynamic random access memory (DRAM) or the like. The image memory 93 can temporarily store a plurality of frames of captured images sequentially output from the camera head 5.

The processing module 94 sequentially processes the captured images received via the communication unit 92. As illustrated in FIG. 2, the processing module 94 includes a memory controller 941 and an image processing unit 942.

The memory controller 941 reads the setting information stored in the register 96 via the selector 95, and controls writing of the captured image to the image memory 93 and reading of the captured image from the image memory 93 according to the setting information.

The image processing unit 942 reads the image processing parameter stored in the register 96 via the selector 95, sequentially receives the image processing parameter via the communication unit 92 using the image processing parameter, and performs image processing on the captured image read from the image memory 93 by the memory controller 941. In addition, the image processing unit 942 generates a display image (a video signal for display) for displaying the captured image after performing the image processing. Then, the image processing unit 942 outputs the display image to the display device 7. As a result, the display image is displayed on the display device 7.

As illustrated in FIG. 2, the image processing unit 942 includes first to fourth image processing blocks 942A to 942D that perform image processing in parallel on each input image. In the first image processing block 942A, the maximum amount of data that can be processed is the data amount of an image of which the number of pixels is HD. The same applies to the other second to fourth image processing blocks 942B to 942D.

Here, examples of the image processing performed by the first to fourth image processing blocks 942A to 942D include optical black subtraction processing, demosaic processing, white balance adjustment processing, digital gain processing (processing of multiplying a digital signal by a digital gain for amplifying the digital signal), noise reduction processing, color correction processing, gamma correction processing, color enhancement processing, contour enhancement processing, enlargement processing, color tone change processing, YC processing of converting an RGB signal (captured image) into a luminance color difference signal (Y, Cb/Cr SIGNAL), and the like.

Note that detailed functions of the processing module 94 (the memory controller 941 and the image processing unit 942) will be described in “Functions of Processing Module” described later.

The selector 95 corresponds to a selector according to the present disclosure. The selector 95 selects at least one of the first and second banks 961 and 962 in the register 96 and determines processing information indicating processing contents by the processing module 94. In the present embodiment, the selector 95 selects one of the first and second banks 961 and 962 and determines processing information indicating processing contents by the processing module 94. More specifically, the selector 95 individually selects a bank for setting processing information to the memory controller 941 and the first to fourth image processing blocks 942A to 942D of the first and second banks 961 and 962 by performing hardware processing.

Note that a detailed function of the selector 95 will be described in “Function of Selector” described later.

The register 96 stores processing information indicating processing contents by the processing module 94. As illustrated in FIG. 2, the register 96 includes first and second banks 961 and 962 which are storage areas of the register 96.

The first bank 961 corresponds to a storage unit according to the present disclosure. The first bank 961 stores processing information corresponding to the normal light image (4K image).

The second bank 962 corresponds to a storage unit according to the present disclosure. The second bank 962 stores processing information corresponding to a fluorescence image (HD image).

Here, the processing information includes setting information used by the memory controller 941 and image processing parameters required when image processing is performed by the image processing unit 942. In the present embodiment, the first and second banks 961 and 962 store the image processing parameters themselves as values related to the image processing parameters according to the present disclosure. The setting information includes the image size of the captured image, the writing position of the captured image in the image memory 93, and the writing and reading sizes in and from the image memory 93.

Hereinafter, the processing information, the setting information, and the image processing parameter stored in the first bank 961 will be referred to as first processing information, first setting information, and a first image processing parameter, respectively. The processing information, the setting information, and the image processing parameters stored in the second bank 962 are referred to as second processing information, second setting information, and a second image processing parameter.

The control unit 97 is realized by executing various programs stored in the storage unit 90 by a controller such as a CPU or a micro processing unit (MPU), and controls the operations of the light source device 3, the camera head 5, and the display device 7 and controls the entire operation of the control device 9. Note that the control unit 97 is not limited to the CPU or the MPU, and may be configured by an integrated circuit such as an application specific integrated circuit (ASIC) or an FPGA.

The control unit 97 has a function of a mode switching unit according to the present disclosure.

Specifically, the control unit (mode switching unit) 97 switches the mode of the image processing system 1 to either a first mode or a second mode in accordance with a user operation on an input unit (not illustrated) provided in the camera head 5 or a user operation on the input unit 98.

Details of the mode of the image processing system 1 will be described in “Function of Selector” described later.

The input unit 98 is configured using an operation device such as a mouse, a keyboard, and a touch panel, and receives a user operation by a user such as an operator. Then, the input unit 98 outputs an operation signal corresponding to the user operation to the control unit 97.

The output unit 99 is configured using a speaker, a printer, or the like, and outputs various types of information.

The storage unit 90 stores a program executed by the control unit 97, information necessary for processing of the control unit 97, and the like.

### Function of Processing Module

Next, functions of the processing module 94 will be described.

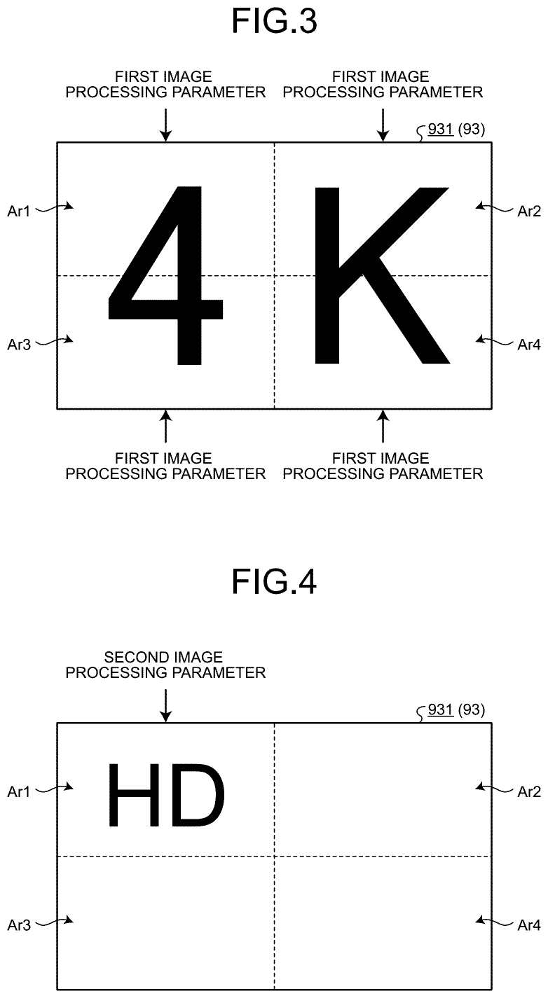

FIGS. 3 and 4 are diagrams illustrating functions of the processing module 94. Specifically, FIGS. 3 and 4 schematically illustrate a specific bank 931 among the plurality of banks in the image memory 93. The bank 931 has a memory capacity corresponding to the data amount of an image having 4K pixels. In FIGS. 3 and 4, the entire area of the bank 931 is equally divided into four first to fourth divided regions Ar1 to Ar4 in a square lattice shape. That is, the first to fourth divided regions Ar1 to Ar4 have memory capacities corresponding to the data amount of the image with the number of pixels of HD. Then, FIG. 3 illustrates a state in which the normal light image (4K image) is written in the bank 931, and characters “4K” are attached to the written position. In addition, FIG. 4 illustrates a state in which the fluorescence image (HD image) is written in the bank 931, and the letter “HD” is attached to the written position.

First, the functions of the processing module 94 (the memory controller 941 and the image processing unit 942) in a case where the captured image received via the communication unit 92 is the normal light image (4K image) will be described with reference to FIG. 3.

The memory controller 941 reads the first setting information (setting information corresponding to the normal light image (4K image)) stored in the first bank 961 in the register 96 via the selector 95. Then, as illustrated in FIG. 3, the memory controller 941 writes the normal light image (4K image) received via the communication unit 92 in the entire area of the bank 931 with the write size corresponding to the first setting information. In addition, the memory controller 941 reads the images (images obtained by dividing the normal light image (4K image)) written in the first to fourth divided regions Ar1 to Ar4, respectively, with the read size (HD size) according to the first setting information. Then, the memory controller 941 outputs the HD size image (image (hereinafter, described as a first divided image) obtained by dividing normal light image (4K image)) read from the first divided region Ar1 to the first image processing block 942A in the image processing unit 942. In addition, the memory controller 941 outputs the HD size image (image (hereinafter, described as a second divided image) obtained by dividing normal light image (4K image)) read from the second divided region Ar2 to the second image processing block 942B in the image processing unit 942. Further, the memory controller 941 outputs the HD size image (image (hereinafter, it is described as a third divided image) obtained by dividing normal light image (4K image)) read from the third divided region Ar3 to the third image processing block 942C in the image processing unit 942. In addition, the memory controller 941 outputs the HD size image (image (hereinafter, it is described as a fourth divided image) obtained by dividing normal light image (4K image)) read from the fourth divided region Ar4 to the fourth image processing block 942D in the image processing unit 942.

Then, the first to fourth image processing blocks 942A to 942D perform image processing in parallel on the input first to fourth divided images (HD images).

Specifically, the first image processing block 942A reads the first image processing parameter (image processing parameter corresponding to the normal light image (4K image)) stored in the first bank 961 in the register 96 via the selector 95. Then, the first image processing block 942A performs image processing on the first divided image by using the first image processing parameter (FIG. 3). Similarly, the second to fourth image processing blocks 942B to 942D also read the first image processing parameter via the selector 95. Then, the second to fourth image processing blocks 942B to 942D respectively perform image processing on the second to fourth divided images by using the first image processing parameters (FIG. 3).

Next, functions of the processing module 94 (the memory controller 941 and the image processing unit 942) in a case where the captured image received via the communication unit 92 is a fluorescence image (HD image) will be described with reference to FIG. 4.

The memory controller 941 reads the second setting information (setting information corresponding to the fluorescence image (HD image)) stored in the second bank 962 in the register 96 via the selector 95. Then, as illustrated in FIG. 4, the memory controller 941 writes the fluorescence image (HD image) received via the communication unit 92 in the first divided region Ar1 which is the writing position corresponding to the second setting information of the bank 931 with the write size corresponding to the second setting information. In addition, the memory controller 941 reads the image (fluorescence image (HD image)) written in the first divided region Ar1 with the read size (HD size) according to the second setting information. Then, the memory controller 941 outputs the HD size image (fluorescence image (HD image)) read from the first divided region Ar1 to the first image processing block 942A in the image processing unit 942.

Then, the first image processing block 942A performs image processing on the input fluorescence image (HD image).

Specifically, the first image processing block 942A reads the second image processing parameters (image processing parameters corresponding to the fluorescence image (HD image)) stored in the second bank 962 in the register 96 via the selector 95. Then, the first image processing block 942A performs image processing on the fluorescence image (HD image) using the second image processing parameter (FIG. 4).

### Function of Selector

Next, a function of the selector 95 will be described.

Hereinafter, regarding the function of the selector 95, a case where the mode of the image processing system 1 is the first mode and a case where the mode is the second mode will be sequentially described.

Here, the first mode is a mode in which the control device 9 sequentially acquires a plurality of types of captured images. In the present embodiment, the first mode is a fluorescence observation mode in which the normal light image and the fluorescence image are generated, and a superimposed image obtained by superimposing the normal light image and the fluorescence image is displayed on the display device 7, or the normal light image and the fluorescence image are displayed on the display device 7.

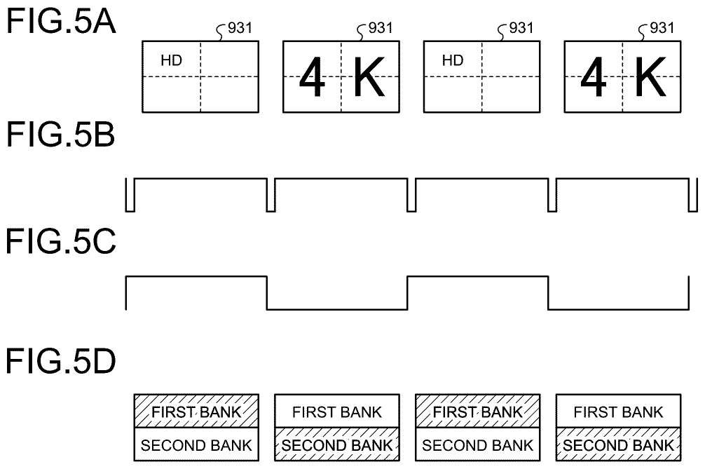

In the first mode, the control unit 97 simultaneously turns on the first and second light sources 31 and 32. Furthermore, the communication unit 54 alternately transmits the normal light image (4K image) and the fluorescence image (HD image) generated by the imaging unit 53 to the communication unit 92 based on a vertical synchronization signal (see FIG. 5B) and a determination signal (see FIG. 5C) output from the control device 9 (reference signal generation unit 91). Note that the determination signal is a signal synchronized with the vertical synchronization signal, and is a signal for determining the type (normal light image (4K image), fluorescence image (HD image)) of the captured image transmitted from the camera head 5 to the control device 9.

The second mode is a mode in which the control device 9 sequentially acquires captured images of the same type. In the present embodiment, the second mode is a normal observation mode in which only the normal light image is generated from the normal light image and the fluorescence image, and the normal light image is displayed on the display device 7. The second mode may be a mode in which only the fluorescence image is generated from the normal light image and the fluorescence image, and the fluorescence image is displayed on the display device 7.

In the second mode, the control unit 97 turns on only the first light source 31 of the first and second light sources 31 and 32. Furthermore, the communication unit 54 sequentially transmits the normal light image (4K image) generated by the imaging unit 53 to the communication unit 92 based on the vertical synchronization signal output from the control device 9.

### Function of Selector in First Mode

FIGS. 5A to 5D are diagrams for describing the function of the selector 95 in the first mode.

Specifically, FIG. 5A is a diagram corresponding to FIGS. 3 and 4, and is a diagram illustrating the captured image written in the bank 931 in the image memory 93. FIG. 5B is a diagram illustrating the vertical synchronization signal output from reference signal generation unit 91. FIG. 5C is a diagram illustrating the determination signal output from reference signal generation unit 91. FIG. 5D is a diagram illustrating a bank selected by the selector 95 of the first and second banks 961 and 962 in the register 96. Note that, in FIG. 5D, the bank of the first bank 961 and 962 that is not selected by the selector 95 is hatched.

As illustrated in FIGS. 5A to 5D, in a case where the selector 95 recognizes that the captured image transmitted from the camera head 5 based on the determination signal is the normal light image (4K image), the selector selects the first bank 961 of the first and second banks 961 and 962 as the bank for setting the processing information (setting information and image processing parameters) to the processing module 94. Meanwhile, in a case where the selector 95 recognizes that the captured image transmitted from the camera head 5 based on the determination signal is the fluorescence image (HD image), the selector selects the second bank 962 of the first and second banks 961 and 962 as the bank for setting the processing information to the processing module 94.

That is, in the first mode, the selector 95 selects the bank storing the processing information corresponding to the type of the captured image from the first and second banks 961 and 962 according to the timing at which the type of the captured image transmitted from the camera head 5 is switched.

### Function of Selector in Second Mode

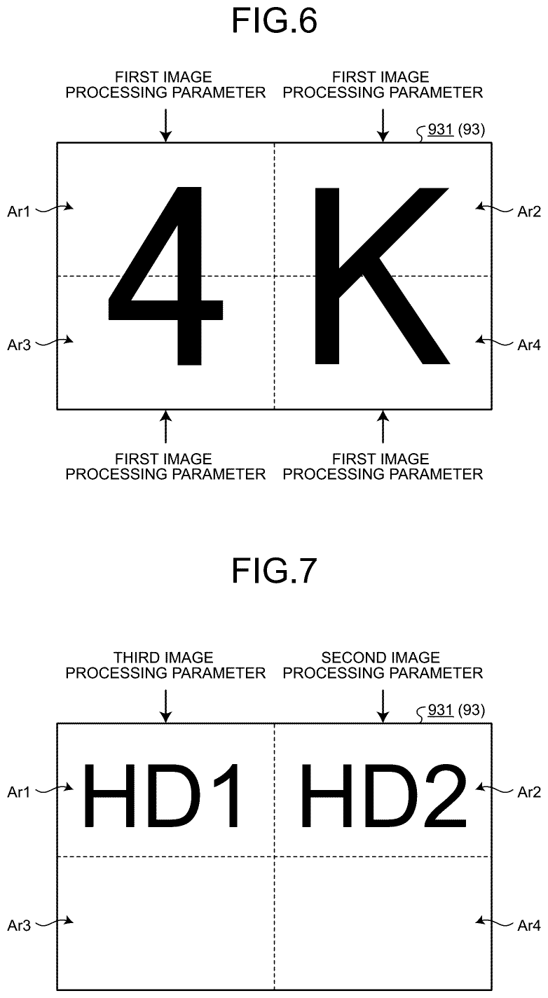

FIG. 6 is a diagram for describing the function of the selector 95 in the second mode. Specifically, FIG. 6 is a diagram corresponding to FIG. 4, and is a diagram illustrating the normal light image (4K image) written in the bank 931 in the image memory 93.

In the second mode, the selector 95 always selects the first bank 961 as the bank for setting the processing information to the processing module 94. As a result, the processing module 94 reads the first processing information (processing information corresponding to the normal light image (4K image)) stored in the first bank 961, and performs processing using the first processing information. That is, the first to fourth image processing blocks 942A to 942D perform image processing on the first to fourth divided images in parallel using the first image processing parameter (FIG. 6).

That is, in the case of the second mode, the selector 95 does not switch the bank to be selected as in the first mode.

According to the present embodiment described above, the following effects are obtained.

The control device 9 according to the present embodiment includes an image processing unit 942 that sequentially performs, for each of a plurality of types of acquired images (normal light image (4K image), fluorescence image (HD image)), image processing using an image processing parameter corresponding to the type of the image for each of the images, first and second banks 961 and 962 that each store the image processing parameter corresponding to the type of the image, and a selector 95 that determines the image processing parameter to be used in the image processing by the image processing unit 942 in accordance with a timing at which the type of the image is switched. More specifically, by performing hardware processing, the selector 95 selects the bank for setting the image processing parameter to the image processing unit 942 from the first and second banks 961 and 962 according to the timing at which the type of the image is switched.

That is, in the control device 9 according to the present embodiment, specific image processing parameters are stored in the first and second banks 961 and 962 in advance, and the banks for which the image processing parameters are set are switched by performing hardware processing. Therefore, it is not necessary to set the image processing parameters by software processing as in the related art, and a load of software can be reduced.

In particular, in the present embodiment, the image processing system according to the present disclosure is applied to the image processing system 1 that is used in the medical field and observes the inside of a living body. Therefore, even in a case where the software runs away, it is possible to switch the bank for setting the image processing parameter by performing the hardware processing, and it is possible to continuously display an appropriate captured image on the display device 7.

### Other Embodiments

Although the embodiments for carrying out the present disclosure have been described so far, the present disclosure should not be limited only by the above-described embodiments.

A configuration of first to eighth modifications described below may be adopted.

### First Modification

FIG. 7 is a diagram illustrating a first modification of the embodiment. Specifically, FIG. 7 is a diagram corresponding to FIGS. 3 and 4.

In the above-described embodiment, the normal observation mode in which the control device 9 sequentially acquires the same type of captured images is adopted as the second mode according to the present disclosure, but the present disclosure is not limited thereto.

As the second mode according to the present disclosure, a mode in which the control device 9 simultaneously acquires a plurality of types of captured images may be adopted.

Specifically, in the second mode according to the first modification, similarly to the first mode, the control unit 97 simultaneously turns on the first and second light sources 31 and 32. Furthermore, the imaging unit 53 generates the normal light image having the number of pixels of HD by thinning reading or pixel accumulation in the first imaging element 531, and generates the fluorescence image (HD image) in the second imaging element 532. Then, the communication unit 54 simultaneously transmits the normal light image (HD image) and the fluorescence image (HD image) to the communication unit 92.

Here, in addition to the first and second banks 961 and 962, the register 96 includes a third bank storing processing information (setting information and image processing parameter corresponding to normal light image (HD image)) indicating processing contents of the processing module 94 corresponding to the second mode. Hereinafter, the processing information, the setting information, and the image processing parameter stored in the third bank will be referred to as third processing information, third setting information, and third image processing parameter, respectively.

Then, in the second mode, the selector 95 always selects the third bank as the bank for setting the setting information in the memory controller 941. As a result, the memory controller 941 reads the third setting information stored in the third bank. Then, the memory controller 941 writes the normal light image (HD image) received via the communication unit 92 in the first divided region Ar1 which is a writing position according to the third setting information, and writes the fluorescence image (HD image) received via the communication unit 92 in the second divided region Ar2 which is a writing position according to the third setting information. Note that, in FIG. 7, the normal light image (HD image) is represented by characters “HD1”, and the fluorescence image (HD image) is represented by characters “HD2”. In addition, the memory controller 941 reads the image (normal light image (HD image)) written in the first divided region Ar1 with the read size (HD size) according to the third setting information, and outputs the image to the first image processing block 942A. Similarly, the memory controller 941 reads the image (fluorescence image (HD image)) written in the second divided region Ar2 with the read size (HD size) according to the third setting information, and outputs the read image to the second image processing block 942B.

In the second mode, the selector 95 always selects the third bank as the bank for setting the image processing parameter to the first image processing block 942A. As a result, the first image processing block 942A performs image processing on the input normal light image (HD image) by using the third image processing parameters (image processing parameters corresponding to the normal light image (HD image)) stored in the third bank (FIG. 7). Hereinafter, the image processing parameters stored in the third bank will be described as third image processing parameters.

Further, in the case of the second mode, the selector 95 always selects the second bank 962 as the bank for setting the image processing parameter to the second image processing block 942B. As a result, the second image processing block 942B performs image processing on the input fluorescence image (HD image) using the second image processing parameter (FIG. 7).

Even in a case where the configuration of the first modification described above is adopted, the same effects as those of the above-described embodiment are obtained.

### Second Modification

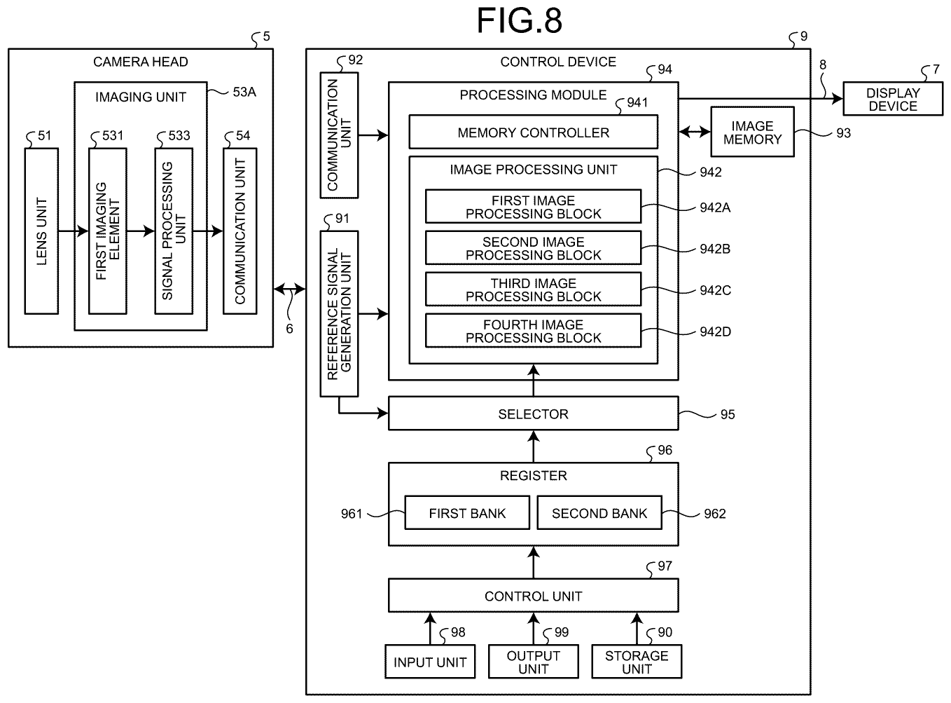

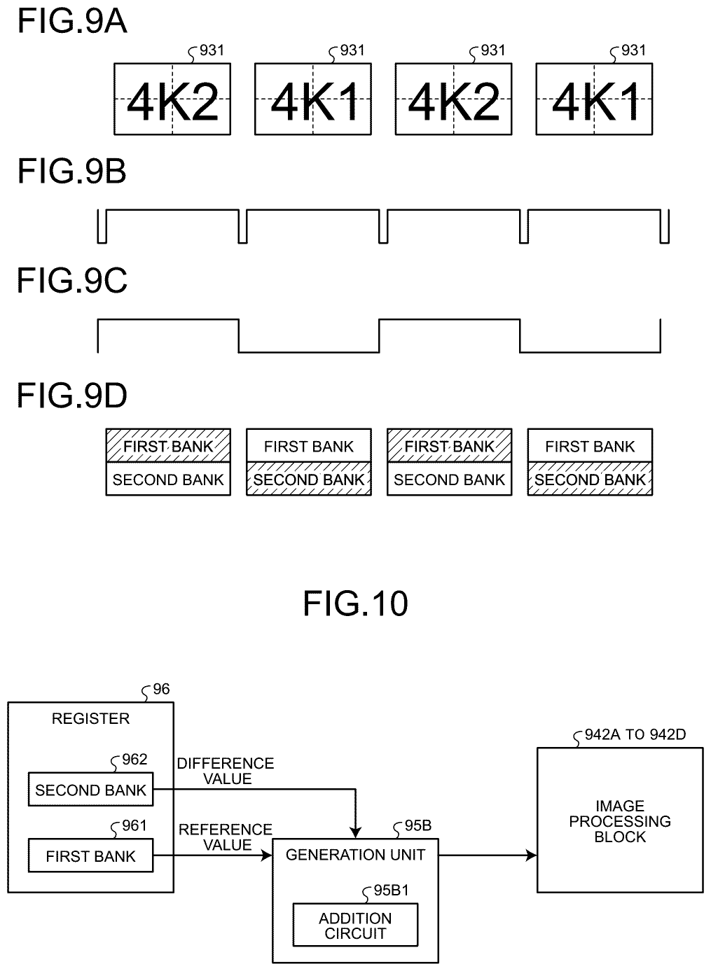

FIGS. 8 and 9A to 9D are diagrams for describing a second modification of the embodiment. Specifically, FIG. 8 is a diagram corresponding to FIG. 2. FIGS. 9A to 9D are diagrams corresponding to FIGS. 5A to 5D. Note that, in FIG. 9A, the normal light image (4K image) is represented by characters “4K1”, and the fluorescence image (4K image) is represented by characters “4K2”.

In the above-described embodiment, the imaging unit 53 includes two imaging elements of the first and second imaging elements 531 and 532, but is not limited thereto, and may include only one imaging element. For example, as illustrated in FIG. 8, an imaging unit 53A according to the second modification does not include the prism 52 and includes only one first imaging element 531.

The first mode according to the second modification is a mode described below.

In the first mode, the control unit 97 turns on the first light source 31 in the first period and turns on the second light source 32 in the second period of the alternately repeated first and second periods.

Furthermore, the first imaging element 531 performs imaging every first and second periods that are alternately repeated in synchronization with the lighting timings of the first and second light sources 31 and 32. As a result, the imaging unit 53A generates the normal light image having 4K pixels by performing imaging in the first period. Furthermore, the imaging unit 53A generates the fluorescence image having 4K pixels by performing imaging in the second period. Then, the communication unit 54 alternately transmits the normal light image (4K image) and the fluorescence image (4K image) generated by the imaging unit 53A to the communication unit 92 based on the vertical synchronization signal (FIG. 9B) and the determination signal (FIG. 9C) output from the control device 9 (reference signal generation unit 91). Note that the determination signal is a signal synchronized with the vertical synchronization signal, and is a signal for determining the type (normal light image (4K image) or fluorescence image (4K image)) of the captured image transmitted from the camera head 5 to the control device 9.

As described above, in the present second modification, the camera head 5 includes one imaging element (first imaging element 531) that outputs different types of images (normal light image (4K image), fluorescence image (4K image)) by capturing the image in a time division manner.

In the second modification, the second bank 962 stores processing information (setting information and image processing parameters) corresponding to a fluorescence image (4K image).

Then, in the first mode, as illustrated in FIGS. 9A to 9D, in a case where the selector 95 recognizes that the captured image transmitted from the camera head 5 based on the determination signal is the normal light image (4K image), the selector selects the first bank 961 of the first and second banks 961 and 962 as the bank for setting processing information to the processing module 94. Meanwhile, in a case where the selector 95 recognizes that the captured image transmitted from the camera head 5 based on the determination signal is a fluorescence image (4K image), the selector selects the second bank 962 of the first and second banks 961 and 962 as the bank for setting the processing information to the processing module 94.

That is, in the first mode, the selector 95 performs hardware processing according to the timing at which the type of the captured image transmitted from the camera head 5 is switched, thereby selecting the bank storing processing information corresponding to the type of the captured image, from the first and second banks 961 and 962.

Even in a case where the configuration of the second modification described above is adopted, the same effects as those of the above-described embodiment are obtained.

### Third Modification

FIG. 10 is a diagram for describing a third modification of the embodiment.

In the above-described embodiment, the first image processing parameter is stored in the first bank 961 and the second image processing parameter is stored in the second bank 962, but the present disclosure is not limited thereto. For example, the following information may be stored in the first and second banks 961 and 962.

The first bank 961 stores a reference value serving as the reference of the image processing parameter as the value related to the image processing parameter according to the present disclosure. In the third modification, the reference value is an image processing parameter (first image processing parameter) corresponding to the normal light image (4K image).

In addition, the second bank 962 stores a difference value that enables generation of a specific image processing parameter by adding the value to the reference value as a value related to the image processing parameter according to the present disclosure. In the third modification, the specific image processing parameter is the image processing parameter (second image processing parameter) corresponding to the fluorescence image (HD image).

Then, in the third modification, a generation unit 95B is provided instead of the selector 95.

The generation unit 95B corresponds to a selector according to the present disclosure. The generation unit 95B includes an addition circuit 95B1 that adds the difference value stored in the second bank 962 to the reference value stored in the first bank 961. Then, in a case where the generation unit 95B recognizes that the captured image transmitted from the camera head 5 based on the determination signal is the normal light image (4K image) in the first mode, the generation unit outputs the reference value (first image processing parameter) stored in the first bank 961 to the image processing unit 942. That is, the normal light image (4K image) corresponds to the first type of image according to the present disclosure. Meanwhile, in a case where the generation unit 95B recognizes that the captured image transmitted from the camera head 5 based on the determination signal is the fluorescence image (HD image) in the first mode, the generation unit adds the difference value stored in the second bank 962 to the reference value stored in the first bank 961 to generate the second image processing parameter, and outputs the second image processing parameter to the image processing unit 942. That is, the fluorescence image (HD image) corresponds to the second type of image according to the present disclosure.

As described above, in the first mode, the generation unit 95B performs hardware processing according to the timing at which the type of the captured image transmitted from the camera head 5 is switched, thereby generating the image processing parameter used in the image processing by the image processing unit 942 using the reference value stored in the first bank 961 and the difference value stored in the second bank 962.

Even in a case where the configuration of the third modification described above is adopted, the same effects as those of the above-described embodiment are obtained.

### Fourth Modification

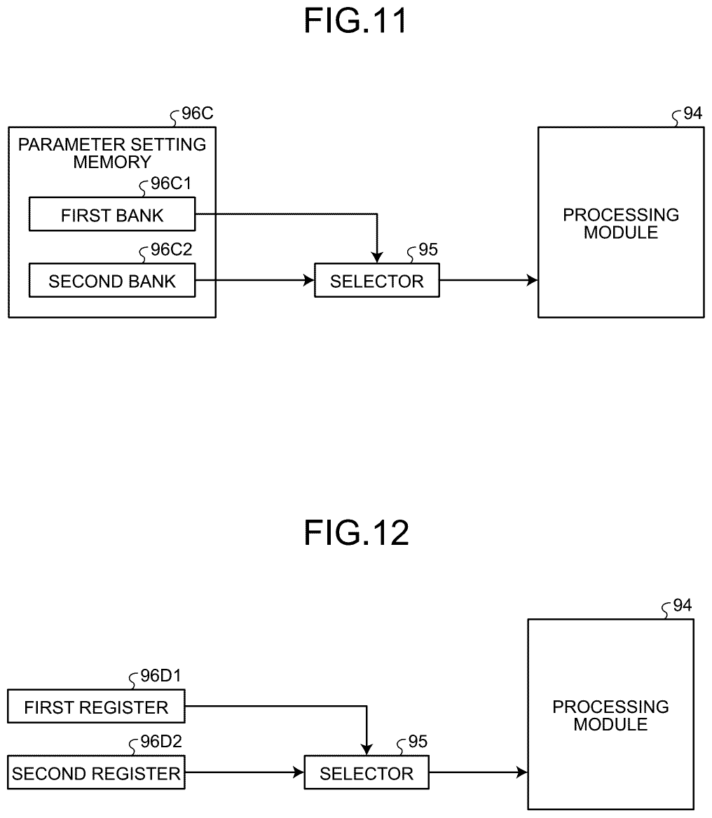

FIG. 11 is a diagram for describing a fourth modification of the embodiment.

In the above-described embodiment, the processing information indicating the processing content of the processing module 94 is stored in the register 96, but the present disclosure is not limited thereto, and the processing information may be stored in a memory. In the fourth modification, a parameter setting memory 96C is provided instead of the register 96.

The parameter setting memory 96C includes first and second banks 96C1 and 96C2 that are storage areas of the parameter setting memory 96C.

The first bank 96C1 corresponds to a storage unit according to the present disclosure. The first bank 96C1 stores processing information (first processing information (first setting information and first image processing parameters)) corresponding to the normal light image (4K image).

The second bank 96C2 corresponds to a storage unit according to the present disclosure. The second bank 96C2 stores processing information (second processing information (second setting information and second image processing parameters)) corresponding to the fluorescence image (HD image).

The function of the selector 95 is similar to that of the above-described embodiment when the first bank 961 is replaced with the first bank 96C1 and the second bank 962 is replaced with the second bank 96C2.

According to the fourth modification described above, the following effects are obtained in addition to the same effects as those of the above-described embodiment.

For example, as the image processing parameter, there is one having a large data capacity, such as an image processing parameter used for gamma correction. By using a memory capable of storing a large amount of data as the storage unit according to the present disclosure instead of a register, it is possible to cope with an image processing parameter having a large data capacity.

### Fifth Modification

FIG. 12 is a diagram for describing a fifth modification of the embodiment.

In the above-described embodiment, the first and second banks 961 and 962 in the register 96 are adopted as the storage unit according to the present disclosure, but the present disclosure is not limited thereto. In the fifth modification, first and second registers 96D1 and 96D2 are provided instead of the register 96.

The first register 96D1 corresponds to a storage unit according to the present disclosure. The first register 96D1 stores processing information (first processing information (first setting information and first image processing parameters)) corresponding to the normal light image (4K image).

The second register 96D2 corresponds to a storage unit according to the present disclosure. The second register 96D2 stores processing information (second processing information (second setting information and second image processing parameters)) corresponding to the fluorescence image (HD image).

The function of the selector 95 is similar to that of the above-described embodiment when the first bank 961 is replaced with the first register 96D1 and the second bank 962 is replaced with the second register 96D2.

Even in a case where the configuration of the fifth modification described above is adopted, the same effects as those of the above-described embodiment are obtained.

### Sixth Modification

The plurality of types of images according to the present disclosure is not limited to the normal light image (4K image) and the fluorescence image (HD image) described in the above-described embodiment.

For example, the fluorescence image (HD image) described in the above-described embodiment is an image acquired by a technology called Infra-Red Imaging (IRI) which is special light observation, but the present disclosure is not limited thereto, and an image acquired by other special light observation (technology called Narrow Band Imaging (NBI), technology called Auto Fluorescence Imaging (AFI), technology called Photodynamic Diagnosis (PDD), and the like) may be adopted.

In addition, the number of the plurality of types of images according to the present disclosure is not limited to two, and may be three or more. At this time, the number of the plurality of storage units according to the present disclosure is matched with the number of the plurality of types of images.

Furthermore, the number of imaging elements according to the present disclosure is not limited to one or two, and may be three or more.

### Seventh Modification

The image processing system according to a seventh modification is an image processing system using a so-called video scope (flexible endoscope) having an imaging unit on a distal end side of the insertion unit. Hereinafter, for convenience of description, the image processing system 1 according to the seventh modification will be referred to as an image processing system 1E.

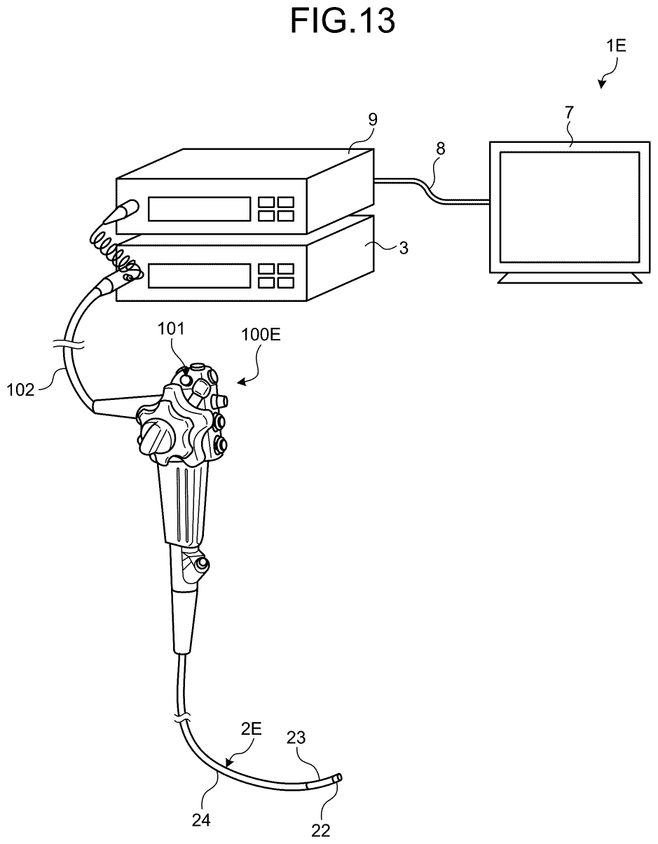

FIG. 13 is a diagram for describing the seventh modification of the embodiment.

As illustrated in FIG. 13, the image processing system 1E includes an endoscope 100E that captures an in-vivo image of an observed region by inserting an insertion unit 2E into a living body and outputs a captured image, a light source device 3 that generates illumination light emitted from a distal end of the endoscope 100E, a control device 9 that processes the captured image output from the endoscope 100E, and a display device 7 that is connected to the control device 9 via a second transmission cable 8 and displays an image based on a video signal processed by the control device 9.

As illustrated in FIG. 13, the endoscope 100E includes the insertion unit 2E having a flexible elongated shape, an operating unit 101 connected to a proximal end side of the insertion unit 2E and receiving various operations, and a universal cord 102 extending in a direction different from a direction in which the insertion unit 2E extends from the operating unit 101 and incorporating various cables connected to the light source device 3 and the control device 9.

As illustrated in FIG. 13, the insertion unit 2E includes a distal end portion 22, a bendable bending portion 23 connected to the proximal end side of the distal end portion 22 and configured by a plurality of bending pieces, and an elongated flexible tube portion 24 connected to the proximal end side of the bending portion 23 and having flexibility.

Although not specifically illustrated, the distal end portion 22 incorporates substantially the same configuration as the camera head 5 described in the above-described embodiment. Then, the captured image captured by the distal end portion 22 (imaging unit) is output to the control device 9 via the operating unit 101 and the universal cord 102.

Even in a case where the configuration of the seventh modification described above is adopted, the same effects as those of the above-described embodiment are obtained.

### Eighth Modification

An image processing system according to an eighth modification is an image processing system using a surgical microscope that enlarges and captures a predetermined visual field area inside a subject (inside a living body) or on a surface of the subject (surface of the living body). Hereinafter, for convenience of description, the image processing system 1 according to the eighth modification will be referred to as an image processing system 1F.

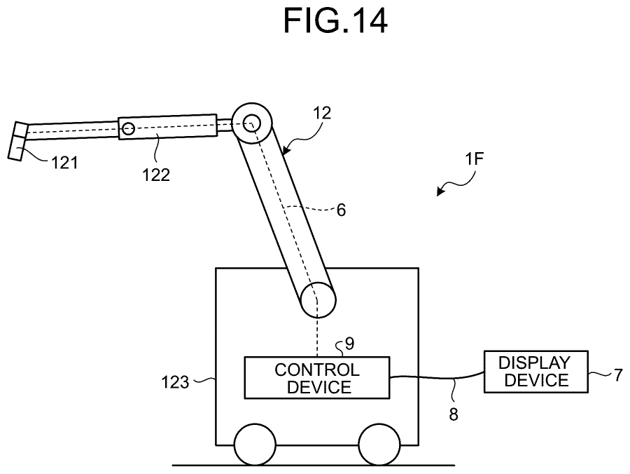

FIG. 14 is a diagram for describing the eighth modification of the embodiment.

As illustrated in FIG. 14, the image processing system 1F includes a surgical microscope 12 that captures an image for observing a subject and outputs a captured image, a control device 9 that processes the captured image output from the surgical microscope 12, and a display device 7 that is connected to the control device 9 via a second transmission cable 8 and displays an image based on a video signal processed by the control device 9.

As illustrated in FIG. 14, the surgical microscope 12 includes a microscope unit 121 that enlarges and captures a minute portion of a subject and outputs a captured image, a support unit 122 that is connected to a proximal end portion of the microscope unit 121 and includes an arm that rotatably supports the microscope unit 121, and a base unit 123 that rotatably holds the proximal end portion of the support unit 122 and is movable on a floor surface.

Then, as illustrated in FIG. 14, the control device 9 is installed on the base unit 123. Although not specifically illustrated, the base unit 123 is also provided with the light source device 3 that generates illumination light to be emitted from the surgical microscope 12 to the subject.

Note that the base unit 123 may be fixed to a ceiling, a wall surface, or the like to support the support unit 122, instead of being movably provided on the floor surface.

Although not specifically illustrated, the microscope unit 121 incorporates substantially the same configuration as the camera head 5 described in the above-described embodiment. Then, the captured image captured by the microscope unit 121 (imaging unit) is output to the control device 9 via the first transmission cable 6 wired along the support unit 122.

Even in a case where the configuration of the eighth modification described above is adopted, the same effects as those of the above-described embodiment are obtained.

Note that the following configurations also belong to the technical scope of the present disclosure.

(1) An image processing device including: an image processing unit that sequentially performs, for each of a plurality of types of acquired images, image processing using an image processing parameter corresponding to a type of the image; a plurality of storage units that each store a value related to the image processing parameter; and a selector that selects at least one of the plurality of storage units according to a timing at which the type of the image is switched and determines the image processing parameter used in the image processing by the image processing unit.

(2) The image processing device according to (1), in which the selector selects one of the plurality of storage units according to the timing at which the type of the image is switched, and determines the image processing parameter used in the image processing by the image processing unit.

(3) The image processing device according to (1) or (2), in which image sizes of the plurality of types of images are different from each other.

(4) The image processing device according to any one of (1) to (3), in which the plurality of types of images includes a 4K image and an HD image.

(5) The image processing device according to any one of (1) to (4), further including a mode switching unit that switches a mode of the image processing device to any one of a first mode for sequentially acquiring a plurality of types of images and a second mode other than the first mode, in which in a case where the mode of the image processing device is the first mode, the selector selects at least one of the plurality of storage units according to a timing at which the type of the medical image is switched and determines the image processing parameter used in the image processing by the image processing unit.

(6) The image processing device according to (5), in which the second mode includes at least one of a mode of sequentially acquiring the same type of medical images and a mode of simultaneously acquiring a plurality of types of medical images.

(7) The image processing device according to any one of (1) to (6), in which each of the plurality of storage units is configured by a register.

(8) The image processing device according to any one of (1) to (6), in which each of the plurality of storage units is configured by a storage area in a register.

(9) The image processing device according to any one of (1) to (6), in which each of the plurality of storage units is configured by a storage area in a memory.

(10) The image processing device according to any one of (1) to (9), in which the selector performs hardware processing to select at least one storage unit that sets the image processing parameter to the image processing unit among the plurality of storage units according to a timing at which the type of the image is switched.

(11) The image processing device according to any one of (1) and (3) to (10), in which when the image is a first type medical image, the selector selects one of the plurality of storage units, and when the image is a second type medical image, the selector sets the image processing parameter to be used in the image processing by the image processing unit using a value related to the plurality of image processing parameters stored in the plurality of storage units.

(12) The image processing device according to (11), in which the plurality of storage units includes a first storage unit that stores a reference value serving as a reference of the image processing parameter, and a second storage unit that stores a difference value that enables generation of the specific image processing parameter by adding the difference value to the reference value, and the selector generates the image processing parameter to be used in image processing by the image processing unit by using the reference value stored in the first storage unit and the difference value stored in the second storage unit according to the timing at which the type of the medical image is switched.

(13) An image processing system including: an imaging device that sequentially outputs a plurality of types of images obtained by imaging; and an image processing device that processes the plurality of types of images sequentially output from the imaging device, in which the image processing device includes an image processing unit that sequentially performs, for each of a plurality of types of acquired images, image processing using an image processing parameter corresponding to a type of the image; a plurality of storage units that each store a value related to the image processing parameter; and a selector that selects at least one of the plurality of storage units according to a timing at which the type of the image is switched and determines the image processing parameter used in the image processing by the image processing unit.

(14) The image processing system according to (13), in which the imaging device includes a plurality of imaging elements that respectively output different types of medical images.

(15) The image processing system according to (13), in which the imaging device includes one imaging element that outputs different types of medical images by performing imaging in a time division manner.

According to the image processing device and the image processing system according to the present disclosure, a load of software can be reduced.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical image processing device comprising:
a plurality of storage units each configured to store a value related to an image processing parameter; and
an image processing unit configured to perform image processing based on the image processing parameter determined by selecting at least one of the plurality of storage units according to a timing at which types of a plurality of types of acquired medical images are switched.

2. The medical image processing device according to claim 1, wherein the plurality of types of medical images includes a medical image obtained by imaging return light of a first wavelength band and a medical image obtained by imaging return light of a second wavelength band different from the first wavelength band.

3. The medical image processing device according to claim 1, wherein the plurality of types of medical images includes a fluorescence image obtained by imaging fluorescence emitted from an observation target when the observation target is irradiated with excitation light, and a white light image obtained by imaging return light from the observation target irradiated with white light.

4. The medical image processing device according to claim 1, further comprising a selector configured to select at least one of the plurality of storage units according to the timing at which the types of the plurality of types of medical images are switched and determines the image processing parameter used in the image processing by the image processing unit, wherein
the selector is configured to
select one of the plurality of storage units according to a timing at which the type of the medical image is switched, and
determine the image processing parameter used in image processing by the image processing unit.

5. The medical image processing device according to claim 1, wherein images sizes of the plurality of types of medical images are different from each other.

6. The medical image processing device according to claim 1, wherein the plurality of types of medical images includes a 4K image and an HD image.

7. The medical image processing device according to claim 1, further comprising:
a selector configured to
select at least one of the plurality of storage units according to the timing at which the types of the plurality of types of medical images are switched, and
determine the image processing parameter used in the image processing by the image processing unit; and
a mode switching unit configured to switch a mode of the image processing device to either a first mode for sequentially acquiring a plurality of types of medical images or a second mode other than the first mode, wherein
in a case where the mode of the image processing device is the first mode, the selector is configured to select at least one of the plurality of storage units according to a timing at which the type of the medical image is switched and determine the image processing parameter used in the image processing by the image processing unit.

8. The medical image processing device according to claim 7, wherein the second mode includes at least one of a mode of sequentially acquiring the same type of medical images and a mode of simultaneously acquiring a plurality of types of medical images.

9. The medical image processing device according to claim 1, wherein each of the plurality of storage units is configured by a register.

10. The medical image processing device according to claim 1, wherein each of the plurality of storage units is configured by a storage area in a register.

11. The medical image processing device according to claim 1, wherein each of the plurality of storage units is configured by a storage area in a memory.

12. The medical image processing device according to claim 1, further comprising
a selector configured to
select at least one of the plurality of storage units according to the timing at which the types of the plurality of types of medical images are switched, and
determine the image processing parameter used in the image processing by the image processing unit, wherein
the selector is configured to perform hardware processing to select at least one storage unit that sets the image processing parameter to the image processing unit among the plurality of storage units according to a timing at which the type of the medical image is switched.

13. The medical image processing device according to claim 1, further comprising
a selector configured to
select at least one of the plurality of storage units according to the timing at which the types of the plurality of types of medical images are switched, and
determine the image processing parameter used in the image processing by the image processing unit, wherein
in a case where the medical image is a first type medical image, the selector is configured to select one of the plurality of storage units, and in a case where the medical image is a second type medical image, the selector is configured to set the image processing parameter to be used in the image processing by the image processing unit using a value related to the plurality of image processing parameters stored in the plurality of storage units.

14. The medical image processing device according to claim 13, further comprising
a selector configured to
select at least one of the plurality of storage units according to a timing at which the types of the plurality of types of medical images are switched, and
determine the image processing parameter used in the image processing by the image processing unit, wherein
the plurality of storage units include:
a first storage unit configured to store a reference value serving as a reference of the image processing parameter; and
a second storage unit configured to store a difference value that enables generation of the specific image processing parameter by adding the difference value to the reference value, and
the selector is configured to generate the image processing parameter to be used in image processing by the image processing unit by using the reference value stored in the first storage unit and the difference value stored in the second storage unit according to the timing at which the type of the medical image is switched.

15. A medical image processing system comprising:
an imaging device configured to sequentially output a plurality of types of medical images obtained by imaging; and
an image processing device configured to process the plurality of types of medical images sequentially output from the imaging device, wherein
the image processing device includes
a plurality of storage units each configured to store a value related to an image processing parameter, and
an image processing unit configured to perform image processing based on the image processing parameter determined by selecting at least one of the plurality of storage units according to a timing at which the types of the plurality of types of acquired medical images are switched.

16. The medical image processing system according to claim 15, wherein the imaging device includes a plurality of imaging elements configured to respectively output different types of medical images.

17. The medical image processing system according to claim 15, wherein the imaging device includes one imaging element configured to output different types of medical images by performing imaging in a time division manner.
